(19) United States
(12) Patent Application Publication
(10) Pub. No.: US 2014/0349961 A1
(43) Pub. Date: Nov. 27, 2014

(54) COMPOSITION FOR ENHANCING INTESTINE METABOLISM

(71) Applicant: TAIWAN HOPAX CHEMS. MFG. CO, LTD., Kaohsiung City (TW)

(72) Inventors: Han-Fen HUANG, Kaohsiung City (TW); Li-Jane HER, Kaohsiung City (TW)

(73) Assignee: TAIWAN HOPAX CHEMS. MFG. CO, LTD., Kaohsiung City (TW)

(21) Appl. No.: 14/286,116

(22) Filed: May 23, 2014

Related U.S. Application Data
(60) Provisional application No. 61/826,614, filed on May 23, 2013.

(51) Int. Cl. A61K 31/737

(52) U.S. Cl. CPC A61K 31/737
USPC 514/55

(57) ABSTRACT

The present invention related to a composition comprising an aqueous soluble-chitosan and a pharmaceutically acceptable carrier. Said composition can be used to increase the defecation rate of an individual. Together with the well known biocompatibility of chitosan, the present invention proves that the aqueous soluble-chitosan may be a potential candidate for enhancing intestinal metabolism.

### BACKGROUND

1. Technical Field

The present invention is related to a composition for enhancing intestine metabolism, especially by using chitosan.

2. Description of Related Art

The intestine is where digestion takes place and also a temporary storage place for digested food residues. Although an intestine is physically inside human body, it is considered as a physiologically outside part of human body in view of human physiology, which means the intestine directly contacts with outer environment and is a critical place that needs well-functional immune system.

Recently, the metabolism of intestine has drawn more and more attention. Researches reveal that the metabolism of intestine is highly related to body health and allergy condition of some individuals. As mentioned above, the intestine is a temporary storage place for digested food residues before being defecated. It is believed that if the digested food residues are retained inside the intestine for too long, the food residues would be further digested by intestinal flora and results in the increase of intestinal flora communities, especially of so called “bad” intestinal flora. Consequently, the amount of bad intestinal flora is increased, the balance inside the intestinal environment is broken, and the activities of the intestinal immune system is consumed so that the intestine metabolism and functionality may be effected, which eventually leads to influence health of the individual concerned. Therefore, although constipation or having excreta reserved in bowel is not a disease, they may be an abnormal or adverse condition that have serious impact on the health of an individual.

In light of the foregoing, it has been acknowledged that the health of an individual is reflected by the well condition in intestine thereof. The intestine metabolism is considered as a key factor that keeps in health. Therefore, there is constantly a need of a composition that is favorable for enhancing intestine metabolism.

### SUMMARY

One object of the present invention is to provide a novel composition has good efficiency in enhancing intestinal metabolism.

In order to achieve the above objects, the present invention provides a composition for enhancing intestinal metabolism, comprising: 0.1 to 80 wt % of an aqueous soluble-chitosan; and 1 to 50 wt % of a pharmaceutically acceptable carrier.

The present invention also provides a method for enhancing intestinal metabolism, comprising: applying a subject in need an effective amount of an aqueous soluble-chitosan.

Preferably, said aqueous soluble-chitosan has a molecular weight of 0.3 to 1500 kDa; more preferably, said aqueous soluble-chitosan has a molecular weight of 0.5 to 300 kDa.

Preferably, said aqueous soluble-chitosan is a chitosan modified by alkyl sultone. More preferably, said alkyl sultone is 1,3-propanesultone, 1,4-propylenesultone, 1,4-butanesultone, 2,4-butanesultone, or a mixture thereof

Preferably, said aqueous soluble-chitosan is a sulfonic acid-modified chitosan.

Preferably, said effective amount is 1 to 500 mg/kgBW.

To sum up, the present invention surprisely found that the aqueous soluble-chitosan has superior effect on increasing the defecation rate of experimental rats. The experimental data supports that the aqueous soluble-chitosan may be a potential candidate for enhancing intestinal metabolism.

### BRIEF DESCRIPTION OF THE DRAWINGS

FIG. 1 shows the comparison of the intestinal tracts of rats in the embodiment 3 of the present invention; (A) ND, (B) HFD, (C) CH, low dosage, (D) CH, high dosage, (E) AS-CH, low dosage, (F) AS-CH, high dosage.

### DETAILED DESCRIPTION

The term of “intestine metabolism” is referred as the efficiency of an intestine tract to digest food and discharge waste. The term of “enhancing intestine metabolism” means to enhance defecation and prevent from constipation or having excreta reserved in bowel.

As known in the field, a high fat diet will cause bad effects on the intestine metabolism of an object. The high correlation between having high fat diet and bad intestine metabolism condition such as constipation or having excreta reserved in bowel has been proved. In this research, the experimental rats were fed with high fat diet to induce the bad intestine metabolism condition and to see if the aqueous soluble-chitosan of the present invention may recover it.

### Embodiment 1

### Preparation of Aqueous Soluble-Chitosan

The aqueous soluble-chitosan of the present invention is chitosan that is modified by alkyl sultone. Examples of alkyl sultone include but not limited to 1,3-propanesultone, 1,4-propylenesultone, 1,4-butanesultone, 2,4-butanesultone, or a mixture thereof. More specifically, the aqueous soluble-chitosan of the present invention is a sulfonic acid-modified chitosan. For example, the aqueous soluble-chitosan is alkyl sulfonic acid-modified chitosan. The alkyl sulfonic acid-modified chitosan may be fabricated by the following procedures:

161 gram of chitosan (with molecular weight of 140,000) was put into a flask, and 700 ml of methanol was added in to obtain a mixture. The mixture was heated at 65 to 67° C., and 122 gram of 1,3-propanesultone was slowly dropped in while stirring. The mixture was kept refluxing for 4 hours after all 1,3-oxathiolane was added in. Then the flask was cooled down to room temperature, and product (alkyl sulfonic acid-modified chitosan) was collected by filtering. The product was washed by methanol from several times and dried overnight in a vacuum oven. The dried product was weighted 282 gram. The yield rate of the alkyl sulfonic acid-modified chitosan was 99.7%.

### Embodiment 2

### Experiment Design of Animal Model

The experiment was conducted by using 4-weeks old weaned Sprague-Dawley rats (purchased from BioLASCO Taiwan Co., Ltd). 64 rats were randomly separated into 8 groups. Each group had 8 rats. The experimental rats were maintained in plastic cages with free access to food and water. The temperature of those cages were kept at 25±1° C., and the day-night cycle was 12 hours per day. For experiments, rats were fed with normal diet (AIN-93G, ICN Biomedicals, Costa Mesa, Calif., USA) or high calorie diet to induce obesity (Modify AIN-93G high fat diet, 20% lipid) for 4 weeks before the administration of aqueous soluble-chitosan. Beginning from the fifth week, the experimental rats were fed with various dosages (10 or 25 mg/kg body weight) of unmodified chitosan and aqueous soluble-chitosan at every Monday, Wednesday, Friday and Saturday. Chitosan used was resolved in sterile water for feeding. One group of normal diet and one group of high calorie diet were instead fed with water as control. The experimental period was 8 to 12 weeks (the experiments were stopped depending on when the body weight of control group and test group show significant difference). The body weight and feeding amount (food intake) of the animals under experiments were measured and recorded every week.

The experimental animals were to be sacrificed by applying carbon dioxide after 12 weeks. Before sacrificing, those animals were starved for 12 hours. Rats' blood and intestinal tract were collected for further analysis.

### Embodiment 3

### Experimental Results

### [Blood Sugar Analysis]

After starvation for 12 hours, the experimental animals were anesthetized by ether. Then the blood was collected from abdominal aorta for analyzing the blood sugar level by enzymatic method and colorimetry method. The results are showed in the following Table 1 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 1
Effects of AS-CH on the blood sugar in HFD rats

| | Blood sugar |
|---|---|
| | mg/dL |
| ND | 163 ± 27.7 |
| HFD | 185 ± 19.6 |
| AS-CH or CH | |
| CH (L) | 172 ± 15.4 |
| CH (H) | 173 ± 23.0 |
| AS-CH (L) | 191 ± 28.7 |
| AS-CH (H) | 168 ± 37.9 |

SD rat was orally administered with various dosages AS-CH (10 or 25 mg/kg BW) for 8 wks. Data is expressed as means ± SD (n = 8).

### [Hepatic & Kidney Function Analysis]

The AST, ALT, creatinine, uric acid were detected by enzymatic method and colorimetry method for determining the hepatic function. The results are showed in the following Table 2 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 2
Effects of AS-CH on the function
of hepatic and kidney in HFD rats

| | AST | ALT | Creatinine | Uric acid |
|---|---|---|---|---|
| | U//L | | mg/dLc | |
| ND | 162 ± 27.7 | 47.5 ± 8.01 | 0.53 ± 0.07 | 3.44 ± 0.98 |
| HFD | 145 ± 35.3 | 49.0 ± 7.78 | 0.51 ± 0.08 | 4.56 ± 0.69 |
| AS-CH or | | | | |
| CH | | | | |
| CH (L) | 176 ± 41.6 | 49.6 ± 9.24 | 0.54 ± 0.05 | 4.90 ± 0.88 |
| CH (H) | 159 ± 32.2 | 44.1 ± 7.71 | 0.50 ± 0.08 | 4.27 ± 0.91 |
| AS-CH (L) | 172 ± 34.9 | 66.6 ± 18.99 | 0.54 ± 0.05 | 4.77 ± 0.38 |
| AS-CH (H) | 161 ± 38.1 | 58.1 ± 25.68 | 0.47 ± 0.05 | 4.45 ± 0.71 |

SD rat was orally administered with various dosages AS-CH (10 or 25 mg/kg BW) for 8 wks. Data is expressed as means ± SD (n = 8).

### [Ketone Bodies and Electrolyte Balance Analysis]

After starvation for 12 hours, the experimental animals were anesthetized by ether. Then the blood was collected from abdominal aorta for analyzing the concentration of ketone bodies, Na⁺ ion and K⁺ ion in the blood by enzymatic method and colorimetry method. The results are showed in the following Table 3 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 3
Effects of AS-CH on the electrolyte
balance and ketone bodies in HFD rats

| | Na⁺ | K⁺ | Ketone bodies |
|---|---|---|---|
| | mEq/L* | | nmole |
| ND | 150 ± 3.06^{ab} | 7.73 ± 1.4^{ab} | 0.96 ± 0.34 |
| HFD | 151 ± 1.33^{a} | 6.92 ± 0.5^{ab} | 1.04 ± 0.35 |
| AS-CH or CH | | | |
| CH (L) | 150 ± 0.92^{ab} | 7.23 ± 1.0^{ab} | 1.25 ± 0.43 |
| CH (H) | 149 ± 1.25^{ab} | 8.16 ± 0.7^{a} | 1.02 ± 0.28 |
| AS-CH (L) | 148 ± 1.63^{b} | 8.39 ± 0.3^{a} | 1.08 ± 0.51 |
| AS-CH (H) | 150 ± 1.33^{ab} | 7.30 ± 1.2^{ab} | 0.99 ± 0.13 |

*mEq/L: molar concentration of ion per liter
SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} electrolyte balance are statistically different from each other (p < 0.05).

By summarizing the analysis of the aforesaid Table 1, Table 2 and Table 3, it was noted that the administration of the aqueous soluble-chitosan of the present invention had no effects on blood sugar, the ketone bodies and electrolyte balance in the blood. Also, it was showed that the administration of the aqueous soluble-chitosan of the present invention had no harm on the liver and kidney function of the experimental animals.

[Analysis for Food Intake, Body Weight, and Feed availability]

As mentioned in the aforementioned paragraphs, the body weight and food intake of the experimental animals were recorded regularly. Based on the recorded body weight, the change in body weight was calculated. Moreover, the feed efficiency was also calculated according to the formula: Feed Efficiency=(Weight Gain/Food Intake)×100%. Also, the organ weight was examined.

The results are showed in the following Table 4, Table 5, Table 6, and Table 7 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 4
Effects of AS-CH on the food intake and body weight in HFD rats

| | Food intake * | | Body weight | | |
|---|---|---|---|---|---|
| | (g/day) | | (g) | | |
| | 8 wks | 16 wks | 0 wks | 8 wks | 16 wks |
| ND | 30.8 | 29.4 | 111 ± 8 | 352 ± 18^{c} | 459 ± 36^{c} |
| HFD | 21.5 | 22.6 | 116 ± 5.5 | 520 ± 31^{a} | 753 ± 24.^{a} |
| AS-CH or CH | | | | | |
| CH (L) | 19.9 | 16.9 | 107 ± 10.8 | 421 ± 31^{b} | 585 ± 57^{b} |
| CH (H) | 21.4 | 16.3 | 113 ± 6.1 | 424 ± 31^{b} | 602 ± 51^{b} |
| AS-CH (L) | 21.8 | 16.9 | 115 ± 7.3 | 440 ± 14^{b} | 602 ± 61^{b} |
| AS-CH (H) | 20.1 | 13.5 | 110 ± 15.0 | 432 ± 21^{b} | 598 ± 62^{b} |

* Data was averaged of groups
SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} body weight are statistically different from each other (p < 0.05)

TABLE 5
Effects of AS-CH on the body weight gain percent in HFD rats

| | Body weight gain (%) | | Change percentage (%) |
|---|---|---|---|
| | 8 wks | 16 wks | 16 − 8 wks |
| ND | 0 | 0 | 0 |
| HFD | 48 | 64 | 16 |
| AS-CH or CH | | | |
| CH (L) | 20 | 27 | 8 |
| CH (H) | 20 | 31 | 11 |
| AS-CH (L) | 25 | 31 | 6 |
| AS-CH (H) | 23 | 30 | 8 |

SD rat was orally administered with various dosages AS-CH (10 or 25 mg/kg BW) for 8 wks. Data is expressed as means ± SD (n = 8).

TABLE 6
Effects of AS-CH on the feed bioavailability in HFD rats

| | Feed bioavailability |
|---|---|
| | % |
| ND | 363.9 |
| HFD | 1029.8 |
| AS-CH or CH | |
| CH (L) | 967.5 |
| CH (H) | 1091.2 |
| AS-CH (L) | 965.2 |
| AS-CH (H) | 1232.4 |

SD rat was orally administered with various dosages AS-CH (10 or 25 mg/kg BW) for 8 wks. Data is expressed as means ± SD (n = 8).

TABLE 7
Effects of AS-CH on the organ weight in HFD rats

| | Heart | Liver | Spleen | Kidney |
|---|---|---|---|---|
| | % of body weight | | | |
| ND | 0.29 ± 0.02 | 2.88 ± 0.09^{b} | 0.14 ± 0.02 | 0.69 ± 0.01 |
| HFD | 0.25 ± 0.03 | 3.45 ± 0.23^{a} | 0.10 ± 0.01 | 0.62 ± 0.04 |
| AS-CH or | | | | |
| CH | | | | |
| CH (L) | 0.29 ± 0.02 | 2.90 ± 0.06^{b} | 0.12 ± 0.02 | 0.62 ± 0.09 |
| CH (H) | 0.27 ± 0.02 | 3.18 ± 0.19^{ab} | 0.14 ± 0.02 | 0.62 ± 0.04 |
| AS-CH (L) | 0.28 ± 0.02 | 2.97 ± 0.09^{b} | 0.13 ± 0.02 | 0.60 ± 0.02 |
| AS-CH (H) | 0.29 ± 0.03 | 2.94 ± 0.20^{b} | 0.12 ± 0.02 | 0.64 ± 0.07 |

SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} organs weight are statistically different from each other (p < 0.05).

The above results indicated that the administration of the aqueous soluble-chitosan of the present invention did not cause significant change in food intake, body weight gain, feed bioavailability and organ weight.

### [Analysis for Intestinal Length and Diarrhea Score]

After scarified, the intestinal tracks of rats were cut for measuring the length. Also, the diarrhea score was examined according to Melgar et al., (2005) by using the following standard table (Table 8).

TABLE 8
Standard table for diarrhea score examination

| Score | Diarrhea Degree |
|---|---|
| 0 | Solid and well-formed stools |
| 1 | Slightly soft stools |
| 2 | Soft stools |
| 3 | Watery stools |

The results are showed in the following Table 9 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 9
Effects of AS-CH on the intestinal physiology in HFD rats

| | Intestinal length (cm) | Diarrhea score (%) |
|---|---|---|
| ND | 18.19 ± 1.12^{a} | 0.00 ± 0.00 |
| HFD | 16.29 ± 0.98^{ab} | 0.00 ± 0.00 |
| AS-CH or CH | | |
| CH (L) | 15.20 ± 1.3^{b} | 0.00 ± 0.00 |
| CH (H) | 16.46 ± 1.87^{ab} | 0.00 ± 0.00 |
| AS-CH (L) | 16.76 ± 1.96^{ab} | 0.00 ± 0.00 |
| AS-CH (H) | 17.36 ± 1.81^{ab} | 0.00 ± 0.00 |

SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} intestinal physiology are statistically different from each other (p < 0.05).

The result showed that the aqueous soluble-chitosan of the present invention has no effects on the intestinal length and did not cause any diarrhea.

### [Analysis for Stool Volume and Defecation Rate]

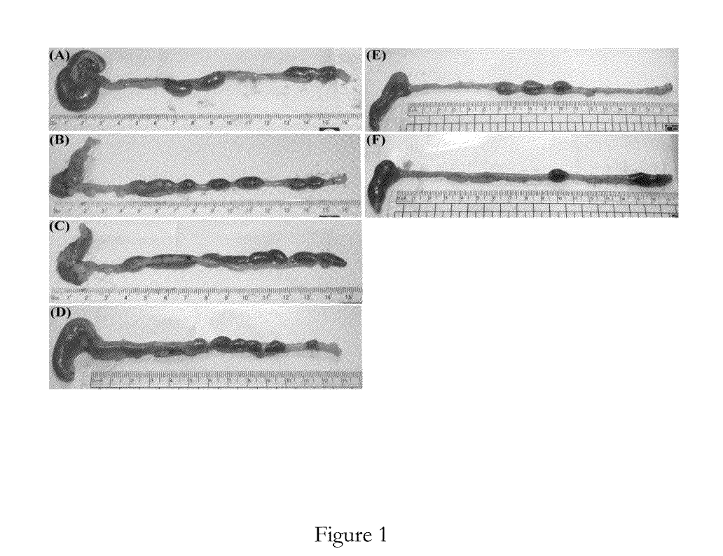

The stool defecated by rats were weighted and recorded, and the defecation rate was calculated by formula: (Stool Volume/Food Intake)×100%. The results are showed in the following Table 10 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)). Also the intestinal tracks cut were pictured as showed in FIG. 1.

TABLE 10
Effects of AS-CH on the stool volume
and defecation rate in HFD rats

| | Stool volume | Defecation rate |
|---|---|---|
| | (g/day/animal) | (% of HFD group) |
| ND | 6.5 | 81 |
| HFD | 6.1 | 100 |
| AS-CH or CH | | |
| CH (L) | 4.2 | 91 |
| CH (H) | 4.2 | 96 |
| AS-CH (L) | 4.9 | 108 |
| AS-CH (H) | 4.5 | 122 |

*Defecation rate = (stool volume/food intake) × 100. Data was shown by taking HFD group as 100%.
SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} intestinal physiology are statistically different from each other (p < 0.05).

According to FIG. 1, more stools were observed to be retrained in the intestinal tract of rats under high fat diet; whereas the intestinal tracks of rats fed with the aqueous soluble-chitosan of the present invention were cleaner or had fewer stools retrained. This observation was consistent with the defecation rate showed in Table 10; wherein the defecation rate was significantly increased in rats fed with the aqueous soluble-chitosan of the present invention.

Those having ordinary skill in the art can understand various modifications according to the disclosed embodiments without departing from the spirit of the present invention. Therefore, the above-recited embodiments shall not be used to limit the present invention but shall intend to cover all modifications under the spirit and scope of the present invention along with the attached claims.

What is claimed is:

1. A composition for enhancing intestinal metabolism, comprising:
0.1 to 80 wt % of an aqueous soluble-chitosan; and
1 to 50 wt % of a pharmaceutically acceptable carrier.

2. The composition according to claim 1, wherein said aqueous soluble-chitosan has a molecular weight of 0.3 to 1500 kDa.

3. The composition according to claim 1, wherein said aqueous soluble-chitosan is a chitosan modified by alkyl sultone.

4. The composition according to claim 1, wherein said alkyl sultone is 1,3-propanesultone, 1,4-propylenesultone, 1,4-butanesultone, 2,4-butanesultone, or a mixture thereof.

5. The composition according to claim 1, wherein said aqueous soluble-chitosan is a sulfonic acid-modified chitosan.

6. A method for enhancing intestinal metabolism, comprising: applying a subject in need an effective amount of an aqueous soluble-chitosan.

7. The method according to claim 6, wherein said aqueous soluble-chitosan has a molecular weight of 0.3 to 1500 kDa.

8. The method according to claim 6, wherein said aqueous soluble-chitosan is a chitosan modified by alkyl sultone.

9. The method according to claim 8, wherein said alkyl sultone is 1,3-propanesultone, 1,4-propylenesultone, 1,4-butanesultone, 2,4-butanesultone, or a mixture thereof.

10. The method according to claim 6, wherein said aqueous soluble-chitosan is a sulfonic acid-modified chitosan.

11. The method according to claim 3, wherein said effective amount is 1 to 500 mg/kgBW.
